(19) United States
(12) Patent Application Publication
(10) Pub. No.: US 2026/0114984 A1
(43) Pub. Date: Apr. 30, 2026

(54) COATED BILIARY STENT

(71) Applicant: BOSTON SCIENTIFIC SCIMED, INC., Maple Grove, MN (US)

(72) Inventors: Rosangel Aimee Ramos Espinoza, Boston, MA (US); Colby Harris, Norfolk, MA (US); David Robert Wulfman, Minneapolis, MN (US); Justin Theodore Nelson, Vadnais Heights, MN (US); Danielle Frankson, Dayton, MN (US); Boxin Tang, St. Paul, MN (US); Riley Duryea, Shakopee, MN (US)

(73) Assignee: BOSTON SCIENTIFIC SCIMED, INC., Maple Grove, MN (US)

(21) Appl. No.: 19/370,380

(22) Filed: Oct. 27, 2025

Related U.S. Application Data
(60) Provisional application No. 63/712,885, filed on Oct. 28, 2024.

(51) Int. Cl. A61F 2/04; A61L 31/04; A61L 31/06; A61L 31/14; A61M 27/00

(52) U.S. Cl. CPC A61F 2/04; A61L 31/048; A61L 31/06; A61L 31/145; A61M 27/008; A61F 2002/041; A61F 2210/0076; A61F 2220/0008; A61L 2430/22; A61M 2205/0238; A61M 2210/1042

(57) ABSTRACT

A biliary stent includes an expandable stent body that is expandable from a pre-deployment collapsed configuration to a post-deployment expanded configuration and a spun coating that is disposed over at least part of the expandable stent body. The spun coating includes a plurality of fibers defining interstices therebetween. The spun coating is adapted to impact tissue ingrowth. The spun coating may be adapted to encourage tissue ingrowth. The spun coating may be adapted to discourage tissue ingrowth.

### CROSS-REFERENCE TO RELATED APPLICATIONS

The application claims the benefit of U.S. Provisional Patent Application Ser. No. 63/712,885, filed on Oct. 28, 2024, the disclosure of which is incorporated herein by reference.

### TECHNICAL FIELD

The present disclosure relates generally to methods and apparatuses for various ailments. More particularly, the disclosure relates to different configurations and methods of manufacture and use of a stent such as a biliary stent.

### BACKGROUND

Implantable stents are devices that are placed in a body structure, such as a blood vessel, esophagus, trachea, biliary tract, colon, intestine, stomach or body cavity, to provide support and to maintain patency of the structure. Biliary stents are stents that are placed within the bile duct. Pancreatic stents are stents that are placed within the pancreas. These devices are manufactured by any one of a variety of different manufacturing methods and may be used according to any one of a variety of methods for a variety of applications. Of the known medical devices, delivery systems, and methods, each has certain advantages and disadvantages. There is an ongoing need to provide alternative medical devices and delivery devices as well as alternative methods for manufacturing and using medical devices and delivery devices.

### SUMMARY

The disclosure is directed to several alternative designs, materials and methods of manufacturing medical device structures and assemblies, and the use thereof. An example may be found in a biliary stent. The biliary stent includes an expandable stent body that is expandable from a pre-deployment collapsed configuration to a post-deployment expanded configuration and a spun coating that is disposed over at least part of the expandable stent body. The spun coating includes a plurality of fibers defining interstices therebetween and is adapted to impact tissue ingrowth.

Alternatively or additionally, the spun coating may be adapted to encourage tissue ingrowth.

Alternatively or additionally, the spun coating may have an average interstice dimension that is equal to or greater than about 10 micrometers.

Alternatively or additionally, the spun coating may include two or more distinct layers.

Alternatively or additionally, the spun coating may be disposed over an entirety of an outer surface of the expandable stent body.

Alternatively or additionally, the biliary stent may further include an adhesive layer that is disposed over the spun coating to facilitate securing the biliary stent in position prior to tissue ingrowth.

Alternatively or additionally, the adhesive layer may be patterned.

Alternatively or additionally, the spun coating may be adapted to discourage tissue ingrowth.

Alternatively or additionally, the spun coating may be adapted to encourage bile drainage through the spun coating.

Alternatively or additionally, the spun coating may have an average interstice dimension that is greater than 0.8 nanometers and less than about 10 micrometers.

Alternatively or additionally, the biliary stent may further include a hydrophilic coating that is disposed on fibers forming the spun coating.

Alternatively or additionally, the biliary stent may further include a hydrogel that is disposed within interstices within the spun coating.

Alternatively or additionally, the expandable stent body may include a braided stent body or a knitted stent body.

Alternatively or additionally, the spun coating may be produced by an electrospinning process, a melt spinning process or an air spinning process.

Another example may be found in a biliary stent. The biliary stent includes an expandable stent body that is expandable from a pre-deployment collapsed configuration to a post-deployment expanded configuration and a spun coating that is disposed over at least part of the expandable stent body. The spun coating includes a plurality of fibers defining interstices therebetween that are adapted to limit tissue ingrowth while enabling bile to drain through the spun coating.

Alternatively or additionally, the spun coating may be hydrophilic.

Alternatively or additionally, the interstices may have an average dimension that is greater than 0.8 nanometers and less than about 10 micrometers.

Another example may be found in a biliary stent. The biliary stent includes an expandable stent body that is expandable from a pre-deployment collapsed configuration to a post-deployment expanded configuration. The expandable stent body includes an inner surface that defines a lumen extending therethrough. The biliary stent includes a polymeric layer that is disposed on the inner surface and a hydrophilic hydrogel that is disposed on the polymeric layer.

Alternatively or additionally, the hydrophilic hydrogel may include a combination of (poly(ethylene glycol) methyl ether methacrylate (PEGMA), N-hydroxyethyl acrylamide (HEAA) and 2-(methacryloyloxy)ethyl trimethylammonium chloride (META)).

Alternatively or additionally, the hydrophilic hydrogel may include a combination of acrylamide (AAm)-2-methacryloxyethyl phosphorylcholine (MPC) and zinc methacrylate (ZMA)-derived poly(AAm-MPCZMA).

Alternatively or additionally, the hydrophilic hydrogel may include a poly(vinylpyrrolidone) based hydrophilic polymer cross-linked with one or more of neopentylglycol diacrylate, ethylene glycol diacrylate or poly(ethylene glycol) diacrylate.

Alternatively or additionally, the biliary stent may further include a polydopamine primer layer on the polymeric layer disposed on the inner surface.

The preceding summary is provided to facilitate an understanding of some of the innovative features unique to the present disclosure and is not intended to be a full description. A full appreciation of the disclosure can be gained by taking the entire specification, claims, figures, and abstract as a whole.

### BRIEF DESCRIPTION OF THE DRAWINGS

The disclosure may be more completely understood in consideration of the following description of various examples in connection with the accompanying drawings, in which:

FIG. 1 is a schematic view of an example biliary stent;

FIG. 1A is an enlarged view of a portion of the example biliary stent of FIG. 1;

FIG. 2 is a schematic view of an example laser-cut stent that may be used as part of the example biliary stent of FIG. 1;

FIG. 3 is a schematic view of an example knitted stent that may be used as part of the example biliary stent of FIG. 1;

FIG. 4 is an enlarged view of a portion of the example knitted stent shown in FIG. 3;

FIG. 5 is a schematic view of an example braided stent that may be used as part of the example biliary stent of FIG. 1;

FIG. 5A is an enlarged view of a portion of FIG. 5; and

FIG. 6 is a schematic view of an example biliary stent.

While the disclosure is amenable to various modifications and alternative forms, specifics thereof have been shown by way of example in the drawings and will be described in detail. It should be understood, however, that the intention is not to limit the disclosure to the particular examples described. On the contrary, the intention is to cover all modifications, equivalents, and alternatives falling within the spirit and scope of the disclosure.

### DESCRIPTION

The following description should be read with reference to the drawings, in which like elements in different drawings are numbered in like fashion. The drawings, which are not necessarily to scale, depict examples that are not intended to limit the scope of the disclosure. Although examples are illustrated for the various elements, those skilled in the art will recognize that many of the examples provided have suitable alternatives that may be utilized.

All numbers are herein assumed to be modified by the term “about”, unless the content clearly dictates otherwise. The recitation of numerical ranges by endpoints includes all numbers subsumed within that range (e.g., 1 to 5 includes 1, 1.5, 2, 2.75, 3, 3.80, 4, and 5).

As used in this specification and the appended claims, the singular forms “a”, “an”, and “the” include the plural referents unless the content clearly dictates otherwise. As used in this specification and the appended claims, the term “or” is generally employed in its sense including “and/or”unless the content clearly dictates otherwise.

It is noted that references in the specification to “an embodiment”, “some embodiments”, “other embodiments”, etc., indicate that the embodiment described may include a particular feature, structure, or characteristic, but every embodiment may not necessarily include the particular feature, structure, or characteristic. Moreover, such phrases are not necessarily referring to the same embodiment. Further, when a particular feature, structure, or characteristic is described in connection with an embodiment, it is contemplated that the feature, structure, or characteristic may be applied to other embodiments whether or not explicitly described unless clearly stated to the contrary.

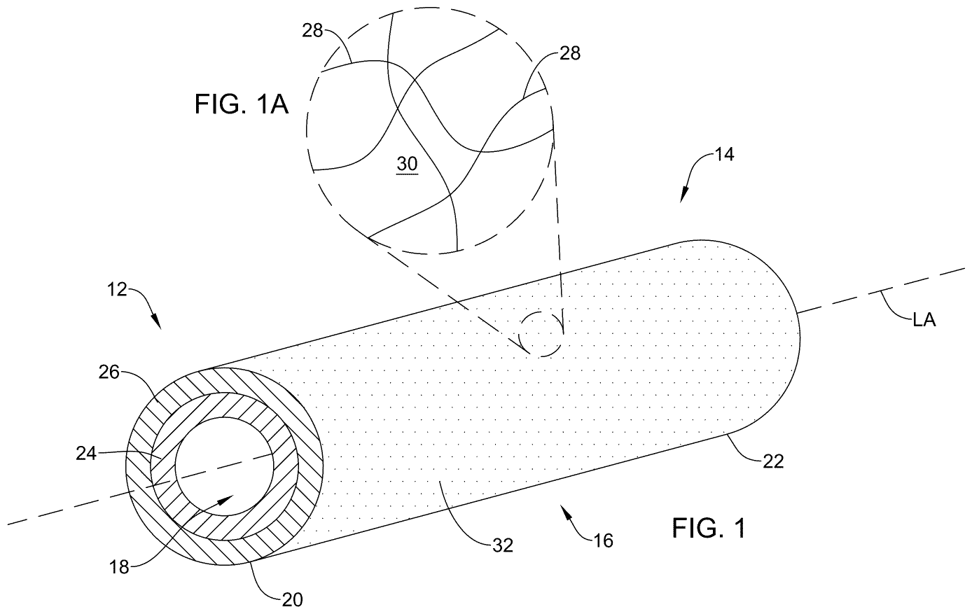

Stents are utilized in a variety of different body lumens, including the vasculature and various parts of the gastrointestinal system, for example. FIG. 1 is a schematic view of an example biliary stent 10 that is adapted for use in body lumens such as the bile duct. In FIG. 1, the example biliary stent 10 is shown as generally tubular, although it is contemplated that the biliary stent 10 may take any cross-sectional shape desired. The biliary stent 10 may be considered as including a first, proximal end region 12, a second, distal end region 14, and an intervening intermediate region 16 extending between the proximal end region 12 and the distal end region 14, recognizing that these designations are arbitrary, and depend on the orientation in which the biliary stent 10 will ultimately be implanted. The biliary stent 10 may be considered as having a constant diameter throughout, including through the proximal end region 12, the distal end region 14 and the intervening intermediate region 16. In some instances, the biliary stent 10 may be considered as having a constant diameter when in a relaxed state. The biliary stent 10 may include a lumen 18 that extends from a proximal end 20 of the biliary stent 10 to a distal end 22 of the biliary stent 10 to allow for the passage of fluids, etc. The biliary stent 10 may be considered as having a longitudinal axis LA. The proximal end region 12 may extend to the proximal end 20 of the biliary stent 10 and the distal end region 14 may extend to the distal end 22 of the biliary stent 10. The biliary stent 10 may be radially expandable from a first radially collapsed configuration (not shown) to a second radially expanded configuration, which may correspond to its relaxed or equilibrium state. In some instances, the radially collapsed configuration may correspond to a pre-deployment configuration while the radially expanded configuration may correspond to a post-deployment configuration. In some instances, the biliary stent 10 may be structured to extend across a stricture and to apply a radially outward pressure to the stricture in a lumen in order to open the lumen and allow for the passage of fluids, etc.

In some instances, the biliary stent 10 includes an expandable stent body 24 that defines the structure of the biliary stent 10. The biliary stent 10 may include an outer layer 26 that does not provide much, or any, of the structure of the biliary stent 10. In some instances, the expandable stent body 24 may provide all or substantially all of the radial strength of the biliary stent 10 while the outer layer 26 may not contribute substantially to the radial strength of the biliary stent 10. In other words, in some instances the radial outward expansion force generated by the biliary stent 10 as the stent 10 transitions from the radially collapsed configuration to the radially expanded configuration may be substantially entirely provided by the expandable stent body 24, while the outer layer 26 does not appreciably contribute to the radially outward expansion force generated by the biliary stent 10. As shown in FIG. 1, the outer layer 26 extends over an entirety of the expandable stent body 24. In some instances, the outer layer 26 may only cover selected portions of the expandable stent body 24. As an example, the outer layer 26 may only cover at least part of the proximal end region 12 and/or at least part of the distal end region 14, and may not cover any of the intermediate region 16.

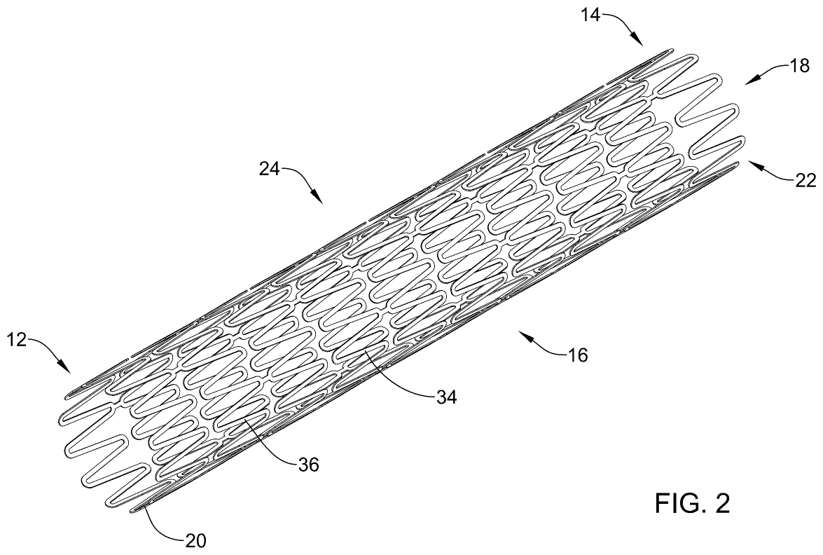

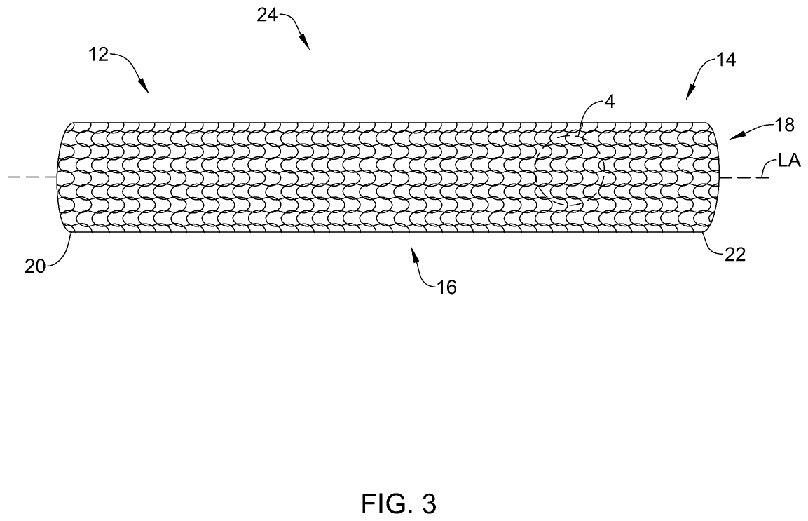

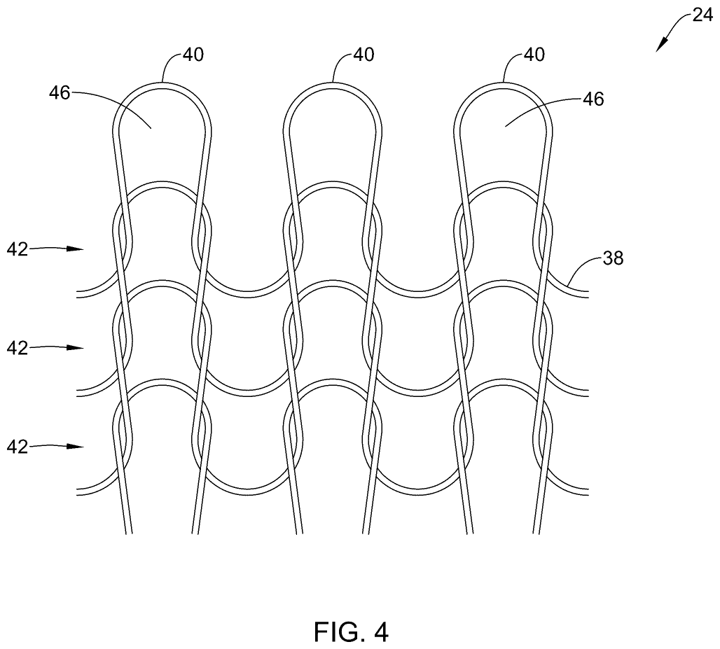

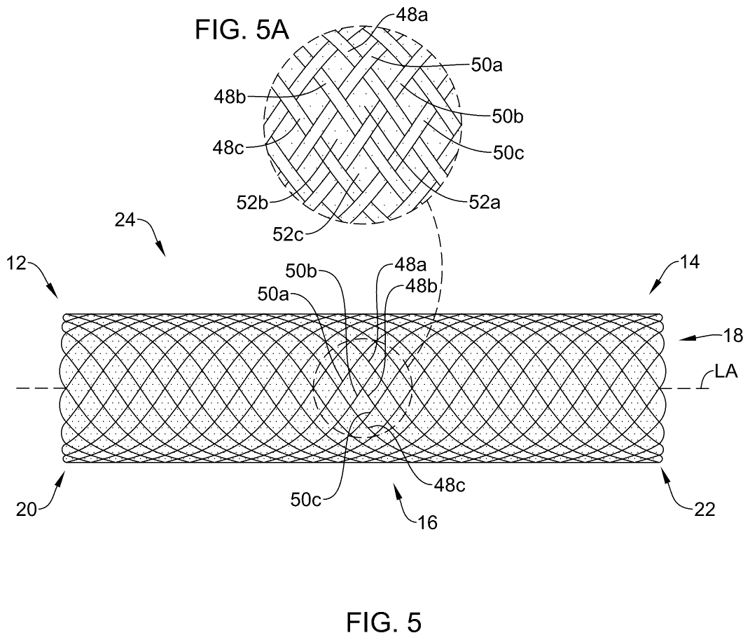

In some instances, the expandable stent body 24 may be a stent formed of a plurality of interconnected struts formed as a monolithic structure from a tubular member. For example, the expandable stent body 24 may be a laser-cut stent, as will be discussed with respect to FIG. 2. A laser-cut stent is a stent that is laser-cut from a cylinder, such as a metal tube. The EPIC™ stents made by Boston Scientific, Corporation are examples of laser-cut stents. In some instances, the expandable stent body 24 may be a knitted stent, which is a stent formed by knitting one or more wires together, as will be discussed with respect to FIGS. 3 and 4. In some instances, a knitted stent may include an open loop knitting pattern. In some instances, a knitted stent may include a twisted loop knitting pattern. The Ultraflex™ stents made by Boston Scientific, Corporation are an example of a knitted stent. In some instances, the expandable stent body 24 may be of a knotted type, such as the Precision Colonic™ stents made by Boston Scientific, Corporation. In other instances, the expandable stent body 24 may be a tubular structure formed of one or more, or a plurality of interwoven wires. In some instances, the expandable stent body 24 may be a braided stent formed of a plurality of braided wires, for example, as will be discussed with respect to FIGS. 5 and 5A. Some exemplary stents including braided filaments include the WallFlex®, WALLSTENT®, and Polyflex® stents, made and distributed by Boston Scientific, Corporation.

The expandable stent body 24 may be formed from a number of different materials such as, but not limited to, metals, metal alloys, shape memory alloys, polymers, as desired, enabling the biliary stent 10 to be expanded into shape when accurately positioned within the body. In some instances, the material may be selected to enable the biliary stent 10 to be removed with relative ease as well. For example, the expandable stent body 24 may be formed from alloys such as, but not limited to, nitinol and Elgiloy®. In some instances, the expandable stent body 24 may be formed from one or more nitinol filaments. In some instances, the expandable stent body 24 may be laser-cut from a nitinol cylinder or tube.

In some instances, the biliary stent 10 may be a self-expanding stent (SES), meaning that the biliary stent 10 will automatically expand into its expanded configuration once any constraints preventing expansion have been removed. In some instances, the biliary stent 10 may not be a self-expanding stent, and thus may rely upon an inflatable balloon or other expandable structure within the lumen 18 in order to cause the biliary stent 10 to expand from its collapsed configuration for delivery to its expanded configuration for deployment in a body lumen.

In some instances, the outer layer 26 may be considered as being a spun coating that may be applied using any of an electrospinning process, a melt spinning process or an air spinning. In each of these processes, a polymer may be applied over the expandable stent body 24 in a manner in which the polymer forms polymer fibers extending over the expandable stent body 24. In particular, electrospinning is a fiber production method that uses electrical force to draw charged threads of polymer solutions for producing nanofibers with diameters ranging from nanometers to micrometers. Electrospinning shares characteristics of both electro-spraying and conventional solution dry spinning of fibers. Melt spinning is a process in which molten thermoplastic polymers are fed at a constant flow rate into a spinning head. The molten polymer is pressed through a spinneret having holes of defined geometry. Air spinning, sometimes referred to as air jet spinning, is a process in which polymeric fibers may be formed using air as a transport mechanism.

Regardless of which mechanism is used for forming the outer layer 26, the outer layer 26 includes a plurality of randomly oriented polymeric fibers that extend over at least part of the expandable stent body 24. FIG. 1A is an enlarged view of a portion of the outer layer 26, showing individual polymer fibers 28. As can be seen, the individual polymer fibers 28 are randomly arranged. While FIG. 1A is a small sample, it will be appreciated that the individual polymer fibers 28 interact to form a number of interstices 30 (one interstice 30 is labeled in FIG. 1A). Because the polymeric fibers 28 are randomly arranged, the interstices 30 will vary in size and shape. In some instances, the sizes of the polymeric fibers 28 and/or the sizes of the interstices 30 may be adjusted, depending on the desired impact of including the outer layer 26 on the expandable stent body 24. In some cases, having larger polymeric fibers 28 may help to facilitate tissue ingrowth while having smaller polymeric fibers 28 may help to discourage tissue ingrowth. For example, having polymeric fibers 28 with a diameter approaching 1000 nanometers can help to facilitate tissue ingrowth while having polymeric fibers 28 with a diameter smaller than about 400 nanometers can help to discourage tissue ingrowth. It will be appreciated that having relatively larger (or relatively smaller) polymeric fibers 28 may influence the corresponding sizes of the resulting interstices 30. Larger polymeric fibers 28 may result in larger interstices 30 and smaller polymeric fibers 28 may result in smaller interstices 30, for example.

In some instances, the sizes of the interstices 30 may be selected in order to facilitate and encourage tissue ingrowth into the outer layer 26 after the biliary stent 10 has been implanted. Tissue ingrowth can assist with helping to anchor the biliary stent 10 in position. In some instances, the sizes of the polymeric fibers 28 and/or the interstices 30 may be selected in order to discourage tissue ingrowth into the outer layer 26 after the biliary stent 10 has been implanted. Because the interstices 30 may be of non-uniform shape and size, it can be helpful to reference an average dimension of the interstices 30. The average dimension may be a numerical average of a particular dimension of each of the interstices 30. The particular dimension may be a width of an interstice 30. The particular dimension may be a height of an interstice 30. The particular dimension may be a depth of an interstice 30. In some instances, the particular dimension of a particular interstice 30 may whatever (height, width, depth) the largest dimension of that particular interstice 30 is.

In some instances, tissue ingrowth into the outer layer 26 may be facilitated or even encouraged by having an average interstice dimension that is large enough to allow tissue cells to fit into the interstices 30. As an example, having an average interstice dimension of at least ten (10) micrometers can encourage tissue ingrowth. In some instances, the outer layer 26 may be functionalized or otherwise have an added adhesive to help anchor the biliary stent 10 in place until tissue ingrowth has occurred. An adhesive layer 32 is schematically illustrated via a dotted pattern on the outer surface of the biliary stent 10. In some cases, the polymeric fibers 28 forming the outer layer 26 may be functionalized with chitosan or transglutaminase in order to promote tissue adhesion. In some instances, functionalization may be achieved by first attaching PEG (polyethylene glycol) to the polymeric fibers 28, followed by applying the chitosan, transglutaminase, or even a combination of chitosan and transglutaminase. In some instances, the adhesive layer 32 may be attached robustly through a physical ingress into the interstices 30 between the individual polymeric fibers 28. In some instances, the adhesive 32 may be attached via chemical reaction, including either chemical cross-linking and/or being grafted into position. In some instances, the adhesive layer 30 may undergo a patterning step in order to improve adhesion to tissue and/or to reduce adhesion to bacterial. The adhesive layer 30 may be patterned in any of a variety of different patterns using direct physical patterning or laser patterning.

In some instances, tissue ingrowth into the outer layer 26 may be discouraged by having an average interstice dimension that is not large enough to allow tissue cells to fit into the interstices 30. As an example, having an average interstice dimension that is less than ten (10) micrometers can help to discourage tissue ingrowth. In some instances, there is a desire for the outer layer 26 to discourage tissue ingrowth yet still permit fluids such as bile to drip through the outer layer 26. As an example, having an average interstice dimension that is greater than about 0.8 nanometers can allow fluids such as bile to drip through while still discouraging tissue ingrowth as long as the average interstice dimension is less than about ten (10) micrometers. In some instances, the outer layer 26 may be chemically modified to promote bile drainage and/or to prevent tissue ingrowth. In some instances, the outer layer 26 may be made hydrophilic. This may be achieved by attaching a hydrophilic polymer such as PEG (poly ethyleneglycol) to the outer layer 26, or to the individual polymeric fibers 28. In some instances, a hydrophilic hydrogel may be incorporated within the interstices 30. This may be done in addition to adding a hydrophilic polymer, or in addition to adding a hydrophilic polymer. The hydrophilic hydrogel may be made of a variety of different polymeric materials that swell when subjected to water. Examples include PEG (polyethylene glycol), PVP (polyvinyl polypyrrolidone), PAA (polyoxyacetic acid, or peracetic acid), and PVA (polyvinyl alcohol). These hydrogels may be cross-linked using a photo-initiator and UV light, for example. Other hydrophilic hydrogels are also contemplated.

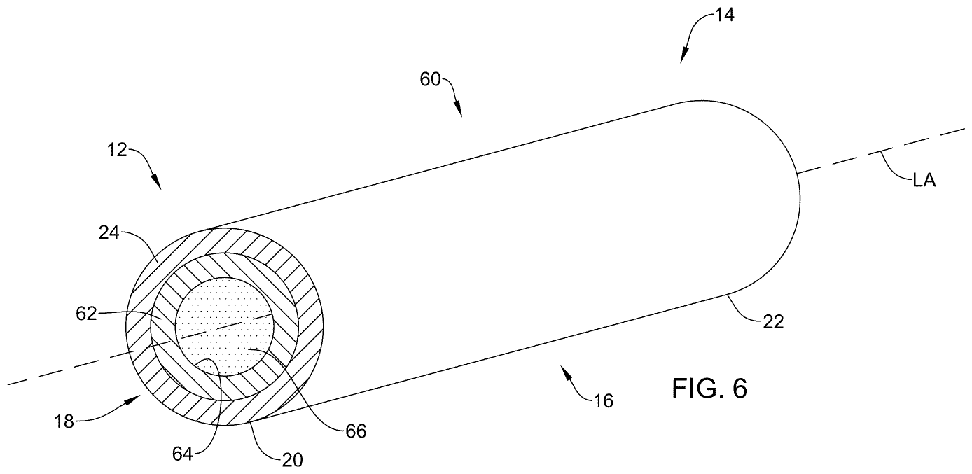

FIG. 2 provides more details regarding the expandable stent body 24 being a laser-cut stent. FIGS. 3 and 4 provide more details regarding the expandable stent body 24 being formed from one or more filaments or wires knitted into an open loop pattern. While not shown, the one or more filaments or wires may also be knitted into a twisted loop knitting pattern. Any of a variety of different knitting patterns may be used. FIGS. 5 and 6 provide more details regarding the expandable stent body 24 being formed by one or more filaments or wires that are braided or woven to form the expandable stent body 24.

FIG. 2 is a schematic view of a monolithic tubular member, formed as a laser-cut stent, that is an example of the expandable stent body 24. As seen in FIG. 2, the expandable stent body 24 includes an expandable monolithic framework 34. The expandable framework 34 may include a number of interconnected struts 36 to form a monolithic mesh-like structure of the expandable framework 34. The expandable framework 34 extends from the proximal end 20 within the proximal end region 12 to the distal end 22 within the distal end region 14. The struts 36 may be adapted to transition from a radially compressed configuration to a radially expanded configuration, for example. In some instances, the struts 36 may be arranged in a suitable pattern, such as a serpentine configuration, a mesh, a fenestrated pattern, or other arrangement. For example, a number of the struts 36 may form a number of alternating peaks and troughs. The struts 36 may have an inner surface and an outer surface, and a thickness extending between the inner surface and the outer surface. The thickness of the struts 36 may be uniform. In some instances, at least some of the struts 36 may vary. As noted, the struts 36 may be formed by laser-cutting a metal cylinder to remove all of the material that is not the struts 36.

FIG. 3 is a schematic view of a knitted stent that is an example of the expandable stent body 24, and FIG. 4 is an enlarged view of a portion thereof. The knitted stent 24 may be considered as having an open loop knitting pattern. The knitted stent 24 extends from the proximal end 20 within the proximal end region 12 to the distal end 22 within the distal end region 14. In some instances, the knitted stent 24 may be produced using an automated weft knitting process that knits a filament 38 into parallel columns 40 of open loops extending longitudinally along the knitted stent 24a and rows 42 of knit stitches extending circumferentially around the knitted stent 24a. The parallel columns 40 run substantially parallel to the longitudinal axis LA of the knitted stent 24 in both the radially expanded, relaxed configuration and an elongated, radially constrained configuration. In some instances, the knitted stent 24 may be formed from a single filament or wire 38, such that each of the rows of knit stiches forming the columns 40 of open loops is formed of only a single filament or wire 38. The knitted stent 24 may be considered as being knitted from a wire or filament 38 that is interwoven with itself, defining interstitial spaces or open cells 46. In some instances, the knitted stent 24 may instead be formed as a spiral knit structure that is formed from a single filament or wire, and instead of distinct rows 42 may have consecutive turns on the spiral. When formed as a spiral knit structure, the knitted stent 24 may have columns 40 that are aligned at a slight angle (e.g., helically arranged) relative to the longitudinal axis LA.

FIG. 5 is a schematic view of a braided stent 24 that is an example of the expandable stent body 24, and FIG. 5A is an enlarged view of a portion thereof. The braided stent 24 extends from the proximal end 20 within the proximal end region 12 to the distal end 22 within the distal end region 14. In some instances, the braided stent 24 includes a first plurality of filaments that extend in a first helical direction and a second plurality of filaments that extend in a second helical direction. The braided stent 24 includes, for example, individual filaments 48a, 48b and 48c, each extending in a first helical direction. The braided stent 24 includes additional filaments (not referenced) extending in the first helical direction. The first helical direction may be considered as extending left to right, or proximal to distal, in a clockwise direction. The braided stent 24 includes, for example, individual filaments 50a, 50b and 50c, each extending in a second helical direction. The braided stent 24 includes additional filaments (not referenced) extending in the second helical direction. The second helical direction may be considered as extending right to left, or distal to proximal, in a clockwise direction.

With reference to FIG. 5A, it can be seen that the individual filaments are braided, i.e., the individual braids extend over and under each other. For example, the individual filament 48a extends under the individual filament 50a, extends over the individual filament 50b, under the individual filament 50c, and so on. Similar relationships exist for each of the individual filaments forming the braided stent 24. It will be appreciated that a cell 52a is formed by the intersections of the individual filaments 48a, 48b, 50a and 50b. A cell 52b is formed by the intersections of the individual filaments 48b, 48c, 50a and 50b. A cell 52c is formed by the intersections of the individual filaments 48b, 48c, 50b and 50c. Each of the cells 52a, 52b and 52c may be considered as being diamond-shaped, having four sides that are roughly equal in length. In some cases, depending on the relative angles at which the first and second helical directions extend, respectively, at least some of the cells may have two sides that are somewhat shorter and two sides that are somewhat longer than the other, for example. The cell 52a and the cell 52c may be considered as being within a single row extending circumferentially about the braided stent 24 while the cell 52b may be considered as being within a neighboring row extending circumferentially about the braided stent 24.

The braided stent 24 can include any number of filaments extending in the first helical direction and any number of filaments extending in the second helical direction. In some instances, the braided stent 24 may have an equal number of filaments extending in the first helical direction and in the second helical direction. In some cases, the braided stent 24 may have a relatively greater number of filaments extending in the first helical direction and a relatively lesser number of filaments extending in the second helical direction. The braided stent 24 may, for example, have a relatively lesser number of filaments extending in the first helical direction and a relatively greater number of filaments extending in the second helical direction. The braided stent 24 may also include one or more filaments that extend in a longitudinal direction, for example. In some cases, the braided stent 24 may have six, seven, eight, nine, ten, eleven, twelve or more filaments extending in the first helical direction and may have six, seven, eight, nine, ten, eleven or twelve or more filaments extending in the second helical direction.

FIG. 6 is a schematic view of an example biliary stent 60 that is adapted for use in body lumens such as the bile duct. In FIG. 6, the example biliary stent 60 is shown as generally tubular, although it is contemplated that the biliary stent 60 may take any cross-sectional shape desired. The biliary stent 60 may be considered as including a first, proximal end region 12, a second, distal end region 14, and an intervening intermediate region 16 extending between the proximal end region 12 and the distal end region 14, recognizing that these designations are arbitrary, and depend on the orientation in which the biliary stent 60 will ultimately be implanted. The biliary stent 60 may be considered as having a constant diameter throughout, including through the proximal end region 12, the distal end region 14 and the intervening intermediate region 16. In some instances, the biliary stent 60 may be considered as having a constant diameter when in a relaxed state. The biliary stent 60 may include a lumen 18 that extends from a proximal end 20 of the biliary stent 10 to a distal end 22 of the biliary stent 60 to allow for the passage of fluids, etc. The biliary stent 60 may be considered as having a longitudinal axis LA. The proximal end region 12 may extend to the proximal end 20 of the biliary stent 60 and the distal end region 14 may extend to the distal end 22 of the biliary stent 60. The biliary stent 60 may be radially expandable from a first radially collapsed configuration (not shown) to a second radially expanded configuration, which may correspond to its relaxed or equilibrium state. In some instances, the radially collapsed configuration may correspond to a pre-deployment configuration while the radially expanded configuration may correspond to a post-deployment configuration. In some instances, the biliary stent 60 may be structured to extend across a stricture and to apply a radially outward pressure to the stricture in a lumen in order to open the lumen and allow for the passage of fluids, etc.

In some instances, the biliary stent 60 includes an expandable stent body 24 that defines the structure of the biliary stent 60. The biliary stent 10 may include an inner layer 62 that does not provide much, or any, of the structure of the biliary stent 60. In some instances, the expandable stent body 24 may provide all or substantially all of the radial strength of the biliary stent 60 while the inner layer 62 may not contribute substantially to the radial strength of the biliary stent 60. In other words, in some instances the radial outward expansion force generated by the biliary stent 60 as the biliary stent 60 transitions from the radially collapsed configuration to the radially expanded configuration may be substantially entirely provided by the expandable stent body 24, while the inner layer 62 does not appreciably contribute to the radially outward expansion force generated by the biliary stent 60.

In some instances, the expandable stent body 24 may be a stent formed of a plurality of interconnected struts formed as a monolithic structure from a tubular member. For example, the expandable stent body 24 may be a laser-cut stent, as discussed with respect to FIG. 2. A laser-cut stent is a stent that is laser-cut from a cylinder, such as a metal tube. The EPIC™ stents made by Boston Scientific, Corporation are examples of laser-cut stents. In some instances, the expandable stent body 24 may be a knitted stent, which is a stent formed by knitting one or more wires together, as discussed with respect to FIGS. 3 and 4. In some instances, a knitted stent may include an open loop knitting pattern. In some instances, a knitted stent may include a twisted loop knitting pattern. The Ultraflex™ stents made by Boston Scientific, Corporation are an example of a knitted stent. In some instances, the expandable stent body 24 may be of a knotted type, such as the Precision Colonic™ stents made by Boston Scientific, Corporation. In other instances, the expandable stent body 24 may be a tubular structure formed of one or more, or a plurality of interwoven wires. In some instances, the expandable stent body 24 may be a braided stent formed of a plurality of braided wires, for example, discussed with respect to FIGS. 5 and 5A. Some exemplary stents including braided filaments include the WallFlex®, WALLSTENT®, and Polyflex® stents, made and distributed by Boston Scientific, Corporation.

The expandable stent body 24 may be formed from a number of different materials such as, but not limited to, metals, metal alloys, shape memory alloys, polymers, as desired, enabling the biliary stent 60 to be expanded into shape when accurately positioned within the body. In some instances, the material may be selected to enable the biliary stent 60 to be removed with relative ease as well. For example, the expandable stent body 24 may be formed from alloys such as, but not limited to, nitinol and Elgiloy®. In some instances, the expandable stent body 24 may be formed from one or more nitinol filaments. In some instances, the expandable stent body 24 may be laser-cut from a nitinol cylinder or tube.

In some instances, the biliary stent 60 may be a self-expanding stent (SES), meaning that the biliary stent 60 will automatically expand into its expanded configuration once any constraints preventing expansion have been removed. In some instances, the biliary stent 60 may not be a self-expanding stent, and thus may rely upon an inflatable balloon or other expandable structure within the lumen 18 in order to cause the biliary stent 60 to expand from its collapsed configuration for delivery to its expanded configuration for deployment in a body lumen.

In some instances, the inner layer 62 may be a polymeric layer such as a silicone layer. While not shown, the biliary stent 60 may also include a silicone layer extending over an outer surface of the expandable stent body 24. In some instances, the silicone layer may be prone to developing biofilms caused by bacteria contacting the silicone. In some cases, a hydrophilic hydrogel 66 may be disposed on an inner surface 64 of the silicone layer 62. The hydrophilic hydrogel 66 is indicated by a dotted pattern formed on the inner surface 64 of the silicone layer 62.

In some instances, the hydrophilic hydrogel 66 may include a combination of (poly(ethylene glycol) methyl ether methacrylate (PEGMA), N-hydroxyethyl acrylamide (HEAA) and 2-(methacryloyloxy)ethyl trimethylammonium chloride (META)). In some cases, the hydrophilic hydrogel 66 may include a combination of acrylamide (AAm)-2-methacryloxyethyl phosphorylcholine (MPC) and zinc methacrylate (ZMA)-derived poly(AAm-MPCZMA). In some cases, the hydrophilic hydrogel 66 may include a poly(vinylpyrrolidone) based hydrophilic polymer that is cross-linked with one or more of neopentylglycol diacrylate, ethylene glycol diacrylate or poly(ethylene glycol) diacrylate. In some cases, while not shown, there may be a polydopamine primer layer disposed on the polymeric layer 62

The materials that can be used for the various components of the biliary stent(s), and the various elements thereof disclosed herein may include those commonly associated with medical devices. For simplicity purposes, the following discussion refers to the apparatus. However, this is not intended to limit the devices and methods described herein, as the discussion may be applied to other elements, members, components, or devices disclosed herein, such as, but not limited to, the medical stent and/or elements or components thereof. In some instances, the apparatus, and/or components thereof, may be made from a metal, metal alloy, polymer (some examples of which are disclosed below), a metal-polymer composite, ceramics, combinations thereof, and the like, or other suitable material.

Some examples of suitable polymers may include polytetrafluoroethylene (PTFE), ethylene tetrafluoroethylene (ETFE), fluorinated ethylene propylene (FEP), polyoxymethylene (POM, for example, DELRIN® available from DuPont), polyether block ester, polyurethane (for example, Polyurethane 85A), polypropylene (PP), polyvinylchloride (PVC), polyether-ester (for example, ARNITEL® available from DSM Engineering Plastics), ether or ester based copolymers (for example, butylene/poly(alkylene ether) phthalate and/or other polyester elastomers such as HYTREL® available from DuPont), polyamide (for example, DURETHAN® available from Bayer or CRISTAMID® available from Elf Atochem), elastomeric polyamides, block polyamide/ethers, polyether block amide (PEBA, for example available under the trade name PEBAX®), ethylene vinyl acetate copolymers (EVA), silicones, polyethylene (PE), MARLEX® high-density polyethylene, MARLEX® low-density polyethylene, linear low density polyethylene (for example REXELL®), polyester, polybutylene terephthalate (PBT), polyethylene terephthalate (PET), polytrimethylene terephthalate, polyethylene naphthalate (PEN), polyetheretherketone (PEEK), polyimide (PI), polyetherimide (PEI), polyphenylene sulfide (PPS), polyphenylene oxide (PPO), poly paraphenylene terephthalamide (for example, KEVLAR®), polysulfone, nylon, nylon-12 (such as GRILAMID® available from EMS American Grilon), perfluoro(propyl vinyl ether) (PFA), ethylene vinyl alcohol, polyolefin, polystyrene, epoxy, polyvinylidene chloride (PVdC), poly(styrene-b-isobutylene-b-styrene) (for example, SIBS and/or SIBS 50A), polycarbonates, polyurethane silicone copolymers (for example, ElastEon® from Aortech Biomaterials or ChronoSil® from AdvanSource Biomaterials), biocompatible polymers, other suitable materials, or mixtures, combinations, copolymers thereof, polymer/metal composites, and the like. In some embodiments the sheath can be blended with a liquid crystal polymer (LCP). For example, the mixture can contain up to about 6 percent LCP.

Some examples of suitable metals and metal alloys include stainless steel, such as 304V, 304L, and 316LV stainless steel; mild steel; nickel-titanium alloy such as linear-elastic and/or super-elastic nitinol; other nickel alloys such as nickel-chromium-molybdenum alloys (e.g., UNS: N06625 such as INCONEL® 625, UNS: N06022 such as HASTELLOY® C-22®, UNS: N10276 such as HASTELLOY® C276®, other HASTELLOY® alloys, and the like), nickel-copper alloys (e.g., UNS: N04400 such as MONEL® 400, NICKELVAC® 400, NICORROS®400, and the like), nickel-cobalt-chromium-molybdenum alloys (e.g., UNS: R30035 such as MP35-N® and the like), nickel-molybdenum alloys (e.g., UNS: N10665 such as HASTELLOY® ALLOY B2®), other nickel-chromium alloys, other nickel-molybdenum alloys, other nickel-cobalt alloys, other nickel-iron alloys, other nickel-copper alloys, other nickel-tungsten or tungsten alloys, and the like; cobalt-chromium alloys; cobalt-chromium-molybdenum alloys (e.g., UNS: R30003 such as ELGILOY®, PHYNOX®, and the like); platinum enriched stainless steel; titanium; platinum; palladium; gold; combinations thereof; or any other suitable material.

In at least some instances, portions or all of the apparatus, and/or components thereof, may also be doped with, made of, or otherwise include a radiopaque material. Radiopaque materials are understood to be materials capable of producing a relatively bright image on a fluoroscopy screen or another imaging technique during a medical procedure. This relatively bright image aids the user of the apparatus in determining its location. Some examples of radiopaque materials can include, but are not limited to, gold, platinum, palladium, tantalum, tungsten alloy, polymer material loaded with a radiopaque filler, and the like. Additionally, other radiopaque marker bands and/or coils may also be incorporated into the design of the apparatus to achieve the same result.

In some instances, a degree of Magnetic Resonance Imaging (MRI) compatibility is imparted into the apparatus and/or other elements disclosed herein. For example, the apparatus, and/or components or portions thereof, may be made of a material that does not substantially distort the image and create substantial artifacts (e.g., gaps in the image). Certain ferromagnetic materials, for example, may not be suitable because they may create artifacts in an MRI image. The apparatus, or portions thereof, may also be made from a material that the MRI machine can image. Some materials that exhibit these characteristics include, for example, tungsten, cobalt-chromium-molybdenum alloys (e.g., UNS: R30003 such as ELGILOY®, PHYNOX®, and the like), nickel-cobalt-chromium-molybdenum alloys (e.g., UNS: R30035 such as MP35-N® and the like), nitinol, and the like, and others.

In some instances, the apparatus and/or other elements disclosed herein may include and/or be treated with a suitable therapeutic agent. Some examples of suitable therapeutic agents may include anti-thrombogenic agents (such as heparin, heparin derivatives, urokinase, and PPack (dextrophenylalanine proline arginine chloromethylketone)); anti-proliferative agents (such as enoxaparin, angiopeptin, monoclonal antibodies capable of blocking smooth muscle cell proliferation, hirudin, and acetylsalicylic acid); anti-inflammatory agents (such as dexamethasone, prednisolone, corticosterone, budesonide, estrogen, sulfasalazine, and mesalamine); antineoplastic/antiproliferative/anti-mitotic agents (such as paclitaxel, 5-fluorouracil, cisplatin, vinblastine, vincristine, epothilones, endostatin, angiostatin and thymidine kinase inhibitors); anesthetic agents (such as lidocaine, bupivacaine, and ropivacaine); anti-coagulants (such as D-Phe-Pro-Arg chloromethyl keton, an RGD peptide-containing compound, heparin, anti-thrombin compounds, platelet receptor antagonists, anti-thrombin antibodies, anti-platelet receptor antibodies, aspirin, prostaglandin inhibitors, platelet inhibitors, and tick antiplatelet peptides); vascular cell growth promoters (such as growth factor inhibitors, growth factor receptor antagonists, transcriptional activators, and translational promoters); vascular cell growth inhibitors (such as growth factor inhibitors, growth factor receptor antagonists, transcriptional repressors, translational repressors, replication inhibitors, inhibitory antibodies, antibodies directed against growth factors, bifunctional molecules consisting of a growth factor and a cytotoxin, bifunctional molecules consisting of an antibody and a cytotoxin); cholesterol-lowering agents; vasodilating agents; and agents which interfere with endogenous vasoactive mechanisms.

Having thus described several illustrative examples of the present disclosure, those of skill in the art will readily appreciate that yet other examples may be made and used within the scope of the claims hereto attached. It will be understood, however, that this disclosure is, in many respects, only illustrative. Changes may be made in details, particularly in matters of shape, size, arrangement of parts, and exclusion and order of steps, without exceeding the scope of the disclosure. The disclosure's scope is, of course, defined in the language in which the appended claims are expressed.

What is claimed is:

1. A biliary stent, comprising:
an expandable stent body expandable from a pre-deployment collapsed configuration to a post-deployment expanded configuration;
a spun coating disposed over at least part of the expandable stent body comprising a plurality of fibers defining interstices therebetween, the spun coating adapted to impact tissue ingrowth.

2. The biliary stent of claim 1, wherein the spun coating is adapted to encourage tissue ingrowth.

3. The biliary stent of claim 1, wherein the spun coating has an average interstice dimension that is equal to or greater than about 10 micrometers.

4. The biliary stent of claim 2, wherein the spun coating comprises two or more distinct layers.

5. The biliary stent of claim 2, wherein the spun coating is disposed over an entirety of an outer surface of the expandable stent body.

6. The biliary stent of claim 2, further comprising an adhesive layer disposed over the spun coating to facilitate securing the biliary stent in position prior to tissue ingrowth.

7. The biliary stent of claim 6, wherein the adhesive layer is patterned.

8. The biliary stent of claim 1, wherein the spun coating is adapted to discourage tissue ingrowth.

9. The biliary stent of claim 8, wherein the spun coating is adapted to encourage bile drainage through the spun coating.

10. The biliary stent of claim 1, wherein the spun coating has an average interstice dimension that is greater than 0.8 nanometers and less than about 10 micrometers.

11. The biliary stent of claim 9, further comprising a hydrophilic coating disposed on fibers forming the spun coating.

12. The biliary stent of claim 9, further comprising a hydrogel disposed within interstices within the spun coating.

13. The biliary stent of claim 1, wherein the expandable stent body comprises a braided stent body or a knitted stent body.

14. The biliary stent of claim 1, wherein the spun coating is produced by an electrospinning process, a melt spinning process or an air spinning process.

15. A biliary stent, comprising:
an expandable stent body expandable from a pre-deployment collapsed configuration to a post-deployment expanded configuration;
a spun coating disposed over at least part of the expandable stent body comprising a plurality of fibers defining interstices therebetween that are adapted to limit tissue ingrowth while enabling bile to drain through the spun coating.

16. The biliary stent of claim 15, wherein the spun coating is hydrophilic.

17. The biliary stent of claim 15, wherein the interstices have an average dimension that is greater than 0.8 nanometers and less than about 10 micrometers.

18. A biliary stent, comprising:
an expandable stent body expandable from a pre-deployment collapsed configuration to a post-deployment expanded configuration, the expandable stent body including an inner surface defining a lumen extending therethrough,
a polymeric layer disposed on the inner surface; and
a hydrophilic hydrogel disposed on the polymeric layer.

19. The biliary stent of claim 18, wherein the hydrophilic hydrogel comprises one of:
a combination of (poly(ethylene glycol) methyl ether methacrylate (PEGMA), N-hydroxyethyl acrylamide (HEAA) and 2-(methacryloyloxy)ethyl trimethylammonium chloride (META)); or
a combination of acrylamide (AAm)-2-methacryloxyethyl phosphorylcholine (MPC) and zinc methacrylate (ZMA)-derived poly(AAm-MPCZMA); or
a poly(vinylpyrrolidone) based hydrophilic polymer cross-linked with one or more of neopentylglycol diacrylate, ethylene glycol diacrylate or poly(ethylene glycol) diacrylate.

20. The biliary stent of claim 18, further comprising a polydopamine primer layer on the polymeric layer disposed on the inner surface.
